SEQUENCE LISTING (12 sequences)

SEQ ID NO: 1 - RNA, 22 nt, Artificial Sequence
  miR-24-3p
uggcucaguucagcaggaacag

SEQ ID NO: 2 - RNA, 6 nt, Artificial Sequence
  portion of miR-24-3p
ggcuca

SEQ ID NO: 3 - RNA, 30 nt, Artificial Sequence
  human BIM site 1
ccccugcaguggaaacugagccagcuaacu

SEQ ID NO: 4 - RNA, 30 nt, Artificial Sequence
  mouse BIM site 1
acccugcagugggaacugagccagcugauu

SEQ ID NO: 5 - RNA, 28 nt, Artificial Sequence
  human BIM site 2
cauauuacuuaucaacugagccaaaugu

SEQ ID NO: 6 - RNA, 28 nt, Artificial Sequence
  mouse BIM site 2
uguuucacucgucaacugagccaaaugu

SEQ ID NO: 7 - RNA, 29 nt, Artificial Sequence
  human PUMA site 1
cccgugaagagcaaaugagccaaacguga

SEQ ID NO: 8 - RNA, 29 nt, Artificial Sequence
  mouse PUMA site 1
ucugugaagagcaaaugagccaaaccuga

SEQ ID NO: 9 - RNA, 29 nt, Artificial Sequence
  human PUMA site 2
gcccagccaucuucccugagccagccggc

SEQ ID NO: 10 - RNA, 29 nt, Artificial Sequence
  mouse PUMA site 2
gcccagccaucuucccugagccgccggcg

SEQ ID NO: 11 - DNA, 14 bp, Artificial Sequence
  LNA miR-24-3p inhibitor 1
tgctgaactgagcc

SEQ ID NO: 12 - DNA, 13 bp, Artificial Sequence
  LNA miR-24-3p inhibitor 2
gctgaactgagcc

(19) United States
(12) Patent Application Publication
(10) Pub. No.: US 2022/0389420 A1
(43) Pub. Date: Dec. 8, 2022

(54) AGENTS FOR THE TREATMENT OF PATIENTS WITH NSCLC AND METHODS TO PREDICT RESPONSE

(71) Applicants: CENTRE NATIONAL DE LA RECHERCHE SCIENTIFIQUE, PARIS CEDEX 16 (FR); UNIVERSITE DE LILLE, LILLE (FR); UNIVERSITE DE NICE SOPHIA ANTIPOLIS, NICE CEDEX 2 (FR); CENTRE HOSPITALIER ET UNIVERSITAIRE DE LILLE, LILLE (FR)

(72) Inventors: Nicolas POTTIER, WASQUEHAL (FR); Christelle CAUFFIEZ, LOMME (FR); Grégoire SAVARY, VALLAURIS (FR); Edmone DEWAELES, LOOS (FR); Cynthia VAN DER HAUWAERT, ENNETIERES EN WEPPES (FR); Bernard MARI, NICE (FR)

(21) Appl. No.: 16/463,886

(22) PCT Filed: Nov. 23, 2017
(86) PCT No.: PCT/EP2017/080266

§ 371 Date: May 24, 2019

(30) Foreign Application Priority Data
Nov. 25, 2016 (EP) 16306563.4

(51) Int. Cl. C12N 15/113; A61K 33/243; A61P 35/00; A61P 11/00; C12Q 1/6886

(52) U.S. Cl. CPC C12N 15/113; A61K 33/243; A61P 35/00; A61P 11/00; C12Q 1/6886; C12N 2310/3231; C12Q 2600/106; C12Q 2600/178; C12Q 2600/156

(57) ABSTRACT

Agents for the treatment of patients having non-small cell lung cancer and methods of diagnostics related include an inhibitor of miR 24 3p, a locked nucleic acid, including methods of predicting response to platinum-based chemotherapy, for patients having, or suspected of having, non-small cell lung cancer.

### INCORPORATION BY REFERENCE

The text file named SEQ_LISTING_AMENDED_2, created on Aug. 23, 2019, and sized 2.65 KB, which contains sequence ID listings, is herein expressly incorporated by reference.

### FIELD OF THE INVENTION

The invention is in the field of the treatment of non-small cell lung cancer. The invention provides agents for the treatment of patients having non-small cell lung cancer and methods of diagnostics related, including methods of predicting response to platinum-based chemotherapy, for patients having, or suspected of having, non-small cell lung cancer.

### BRIEF DESCRIPTION OF THE INVENTION

Lung cancer is a leading cause of death worldwide, resulting in more than 1.3 million deaths per year (Jemal et al., 2008). The majority (>80%) of lung cancers are attributable to tobacco smoke and it is assumed that overall one in nine smokers will develop lung cancer in their lifetime (Jemal et al., 2008). Lung cancer is a primary malignant neoplasm occurring in the bronchus, trachea or lung tissue and can be divided into two distinct main entities: small cell lung cancer (SCLC) and non-small cell lung cancer (NSCLC), which develop from different types of lung cells and behave differently with respect to growth characteristics and spread in patients. This distinction is critical, both clinically and in terms of tumor genetics and biology (Goldstraw et al., 2011).

Non-small cell lung carcinoma (NSCLC) accounts for at least 80% of cases with lung adenocarcinoma (LUAD) being the most common form in many countries (World Health Organization, 2012). At diagnosis, patients with NSCLC can be divided into 4 stages that reflect the extent of the disease and, consequently, treatment. Patients with stage I disease have the best prognosis, with a 5-year survival rate of 60% or better. Stages II and IIIA include patients with either locally or regionally advanced lung cancer. Five-year survival in these patients ranges between 10 and 50%. The final group includes patients with advanced disease with or without distant metastases (stages IIIB and IV). These patients have the worst prognosis with a 5-year survival below 10%. Of note, most patients with early-stage disease will ultimately experience tumor recurrence and die of lung cancer. Finally, the estimated overall 5-year survival for patients suffering of NSCLC is merely 16% (Goldstraw et al., 2011).

Although notable progress has been made in the treatment of NSCLC over the last decade, this malignancy is still associated with poor response rates to therapy as well as a dismal prognosis for most patients (Chang, 2011). Surgery is considered as the key component of therapy for early stage NSCLC and recent guidelines recommend to use a first line platinum-based combination therapy in association with surgery or radiotherapy in either neoadjuvant or adjuvant settings (Friboulet et al., 2013; Goldstraw et al., 2011). Most patients are diagnosed at an advanced stage of NSCLC and are not eligible to surgical resection, and platinum-based combination therapy is the first-line treatment in this group of patients. Variability in NSCLC drug response and toxicity among patients is multifactorial, including (epi)genetic and disease determinants or environmental factors such as hypoxia (Chang, 2011). Therefore elucidating the molecular mechanisms underlying cancer cell resistance to current therapy is critical to pinpointing strategies to prevent these escape routes. While significant progress has been made in the understanding of NSCLC drug resistance, the role of non-coding RNA, and in particular miRNA, is poorly documented. In this context, the inventors have shown the previously unreported role of miRNAs of the miR-24-3p cluster and have derived useful methods and products for the diagnosis and treatment of NSCLC. More specifically, the inventors have shown that miR-24-3p is expressed at higher level in patients with poor prognosis and that inhibition of miR-24-3p could sensitize to platinum-based chemotherapy.

The inventors therefore provide an inhibitor of miR-24-3p, in particular for use in the treatment of a patient, in particular a human patient, and more particularly a human patient having non-small cell lung cancer (NSCLC), in particular lung adenocarcinoma (LUAD), more particularly stage III or IV NSCLC or LUAD. The inhibitor is also provided in combination with a platinum-based chemotherapy agent, and/or is provided for use in a patient (in particular a patient as above) treated with a platinum-based chemotherapy, in particular when said chemotherapy treatment is performed simultaneously with or after the treatment with the inhibitor.

The inventors also provide herein an in vitro method of predicting likelihood of response to platinum-based chemotherapy in a patient, in particular a human patient, and more particularly a human patient having non-small cell lung cancer (NSCLC), in particular lung adenocarcinoma (LUAD), more particularly stage III or IV NSCLC, comprising the steps of (i) obtaining a biological sample from said patient; and (ii) assessing the level of expression of at least one miRNA selected from the group consisting of miR-24-3p, miR-23a-3p and miR 27a-3p. A method with similar steps is also provided to select patients likely to benefit from treatment with an inhibitor of miR-24-3p (possibly in combination with platinum-based chemotherapy)

The inventors also provide an inhibitor of miR-24-3p for use in a patient wherein the expression level of at least one miRNA selected from the group consisting of miR-24-3p, miR-23a-3p and miR-27a-3p was measured, in particular wherein the expression level of said miRNA(s) in said patient was/were found to be equal to or higher than a reference level. The inhibitor is provided in particular for use in such patient in combination with a platinum-based chemotherapy agent, and/or is provided for use in such patient treated with a platinum-based chemotherapy

The inventors also provide platinum-based chemotherapeutic agents for use in a patient, in particular a human patient, and more particularly a human patient having non-small cell lung cancer (NSCLC), in particular lung adenocarcinoma (LUAD), more particularly stage III or IV NSCLC, wherein the expression level of at least one miRNA selected from the group consisting of miR-24-3p, miR-23a-3p and miR-27a-3p was measured in said patient, in particular wherein the expression level of said miRNA(s) were found to be lower in said patient than a reference level.

### DETAILED DESCRIPTION OF THE INVENTION

Methods of Determining Level of Expression of Given miRNAs and Diagnostics Methods

The inventors provide herein methods including a step of assessing, or determining the level of expression of miRNAs in biological samples. Assessing the level of expression refers in particular to direct measurement of the absolute amount of said miRNAs. Assessing the level of expression may also refer to relative measurement of said level, in particular to method steps wherein the amount of said miRNA(s) is determined in relation to the amount of other RNAs, in particular to other miRNAs.

The value may be expressed in any suitable units known to the skilled person. In particular, said units are in linear or logarithmic relation to the amount of miRNA in a cell or in a given amount of sample. Said units may also be in linear or logarithmic relation to the molar ratio or ratio by weight of the miRNA relative to another molecular or biological entity (or other entities) in the sample, in particular one or several proteins (or the total amount of proteins) or one or several RNAs (or the total amount of RNA), more particularly one or several miRNAs. Said units may also relate not directly to the amount of miRNA, but to a parameter that varies with said amount, such as the number of cycles to obtain significant amplification in qPCR-type procedures. Said units may be arbitrary, in the sense that they relate to the amount of miRNAs only in a given experimental procedure which can generally not be compared to amounts determined in independent procedures: the amount must be compared to other amounts (e.g. amounts of other miRNAs) determined in the same procedure and expressed in similar arbitrary units, so a comparison is possible.

In certain embodiments, the diagnostics method of the invention comprises a step of determining the level of expression of at least one miRNA selected from the group consisting of miR-24-3p, miR-23a-3p and miR-27a-3p. miR-24-3p, miR-23a-3p and miR-27a-3p are collectively referred to as “miRNAs of the miR-24-2 cluster”, and “a miRNA of the miR-24-2 cluster” designates at least one (i.e. one, two, or three) miRNA selected from the group of miRNAs of the miR-24-2 cluster. The miRNAs of which the level of expression is determined are referred to herein as the “miRNAs of interest”. This does not include miRNAs of which the amount is determined for calibration or normalization purposes, as detailed below. The three miRNAs of the miR24-2 cluster are expressed simultaneously and form part of the same transcriptional entity.

In certain embodiments, the expression level from one of the miRNAs in the group is tested. In particular embodiments, the expression level of miR-24-3p is tested. In certain embodiments, the expression level of two of the miRNAs is tested. In certain embodiments, the expression level of miR-24-3p and miR-23a-3p, or of miR-24-3p and miR-27a-3p or of miR-23a-3p and miR-27a-3p is tested. In certain embodiments, the expression levels of all three miRNAs of the miR-24-2 cluster are tested.

In certain embodiments wherein the expression levels of several miRNAs are determined, and/or wherein the expression levels of the same miRNA is determined several times, several values may be combined in a single value to be used in particular for comparison (or scoring) purposes. “Combining values”, as used herein, means performing an operation, in particular a mathematical operation, using said values to obtain a combined value, directly related to the original values. In particular, the combined value is the average value of the original values, or the median value, or the sum of the original values. In particular, if several determinations are performed for the same miRNA, a combined value, in particular the average value, of these determinations may be used to represent the expression level for this miRNA. Similarly, the average value of the expression level of several miRNAs of interest may be used. The median value, the sum, or any other combined value, may be used instead of the average value.

In certain embodiments, the expression level is normalized. Normalization is performed using methods known to the skilled person. The level may be normalized in particular in relation to the expression levels of one or several miRNAs or other RNAs (like snRNAs or “spike-in” RNAs). Such miRNAs are preferably unrelated to miR-24-3p and are preferably not related to any of the miRNAs of the miR-24-2 cluster.

In certain embodiments, the diagnostics method of the invention comprises a step of comparing the expression level of the miRNAs of interest with a reference level (or reference expression level).

In certain embodiments, the reference expression level is determined from determinations (“reference determinations”) of the expression level of the same miRNAs, in identical experimental conditions. The expression levels from the reference determinations are preferably submitted to the same numerical transformations as the sample determinations. In particular, expression is normalized using identical rules. Similarly to the expression levels in the sample of interest, the expression levels for the reference determinations, where several such determinations are performed, may be combined. In general, the same operation will be performed on the expression levels and the reference expression levels.

In embodiments where the measured expression level of a miRNA of the miR-24-2 cluster is compared with a reference expression level, said measured expression level may be said to be “higher” than said reference expression level when the measured level is higher than the reference level by any amount, or alternatively the measured level may be said to be “higher” than the reference level only when the difference is sufficient to be significant. In particular the measured expression level may be said to be higher than the reference expression level when the ratio (measured/reference) is greater than 1, or when the ratio is greater than 1.1, preferably when the ratio is greater than 1.5. Conversely, the measured expression level may be said to be lower than the reference expression level (i) when it is not higher according to one of the above criteria or (ii) when it is lower, by any amount, than the reference level or (iii) when it is lower by a significant amount than the reference level, in particular when the ratio measured/reference is lower than 1, or lower than 0.9, or lower than 0.5, or lower than 0.1.

Testing methods provided herein are preferably performed in vitro. Generally speaking, the skilled person is aware of in vitro methods for testing expression levels of miRNA in samples from patients. Guidance may be found in the examples section and in references cited herein.

In certain embodiments, the sample is a tumour sample, i.e. a sample taken from tissue comprising cancerous cells, and more particularly a sample from the primary tumour or from a metastasis. In particular embodiments, the sample is a sample from a lung biopsy. A needle biopsy, or a biopsy obtained by any other method known in the art, may be used. In certain embodiments, the sample is a fluid sample, i.e. a sample from a biological fluid, in particular a blood sample, in particular a serum sample or a plasma sample. In certain embodiments, the sample comprises or consists of circulating tumour cells, i.e. tumour cells found in the bloodstream. The skilled person is aware of methods to isolate (or concentrate) such cells from a blood sample and the sample may be in particular a sample of isolated circulating tumour cells.

In certain embodiments, the reference expression is the expression level in non-tumour cells of the same patient. In particular, the reference expression is the expression level in non-tumour tissue of said patient. In certain embodiments, the reference level is established from expression levels measured in a reference population. In certain embodiments, the reference expression level is established as the average of measurements performed in the reference population. In certain embodiments, the reference expression level is the median expression level in the reference population.

In certain embodiments, the reference population comprises in particular at least 10 individuals, preferably at least 50, more preferably at least 100 and even more preferably at least 250 individuals. In certain embodiments, the reference population is an unselected population, i.e. the individuals in the population are not selected according to any particular criteria. In certain embodiments, the reference population comprises a majority of, or solely, individuals which do not suffer from any diagnosed cancer. In certain embodiments, the reference population comprises a majority of, or solely, individuals which suffer from lung cancer, in particular NSCLC and more particularly LUAD. In certain embodiments, the reference population comprises a majority of, or solely, individuals which suffer from NSCLC, particularly LUAD, and who are either unselected for response to chemotherapy (i.e. response or non-response to platinum-based chemotherapy is not a selection factor) or are selected non-responder patients, i.e. the patients included in the reference population show no response (and/or acquired resistance) to platinum-based chemotherapy.

The diagnostics method of the invention is generally useful to assist in assessing at least one of the condition of a patient, the evolution of said condition, the predictable evolution of said condition, the response to a treatment (i.e. the evolution of the condition when under said treatment), in particular by platinum-based chemotherapy, of a patient and/or the predictable response to said treatment. Methods suitable and/or intended for such uses are provided herein. Such methods are collectively referred to herein as “diagnostics methods”, in the broadest interpretation of the term by the skilled person. In particular, diagnostics methods comprise diagnostics in the narrow meaning of determining the existence and nature of a pathological condition in a patient, as well as prognosis (determining predictable evolution of a condition), staging (determining severity and/or degree of evolution, of a condition), companion diagnostics (determining response, or likelihood of response, to a treatment), predisposition testing, etc.

In preferred embodiments, the patient is a human patient, in particular a patient suffering from non-small cell lung cancer, in particular lung adenocarcinoma. In certain embodiments, the cancer is non-small cell lung cancer (NSCLC). In certain embodiments, the cancer is lung adenocarcinoma (LUAD). In certain embodiments, the cancer is a stage III or IV NSCLC and/or LUAD.

In preferred embodiments, the method is intended to predict increased likelihood of resistance, or increased likelihood of response to platinum-based chemotherapy treatment. Prediction of likelihood (of response and/or resistance to treatment) is intended to mean provision of any piece of information regarding said likelihood, wherein said piece of information could not be obtained by, or comes in addition to information provided by, the clinical status of the patient and any parameters non directly related to the expression of a miRNA of the miR-24-2 cluster. In particular, the “baseline” likelihood of response and/or resistance is established prior to (or independently of) performing the method of the invention, based on clinical observation of the patient, medical imaging, biological tests (not directly related to the expression of a miRNA of the miR-24-2 cluster), etc. and the baseline value (or estimate) obtained is modified (increased, or decreased, with or without a quantitative assessment of the extent of such increase or decrease) taking in account the expression level measured using a method of the invention.

More particularly, the baseline likelihood of response, determined independently of the method of the invention, is increased when said expression level of a miRNA of the miR-24-2 cluster is determined to be lower than a reference value and/or the likelihood of resistance is increased when said expression level is determined to be equal to or higher than a reference value.

In particular, the patient is said to be likely to respond to platinum-based chemotherapy when the likelihood (or probability) of responding to said chemotherapy is higher than the average likelihood of response among patients with cancer, or more specifically among patients with cancers of the same or of a similar type and/or at the same or a similar stage, in particular of patients with NSCLC, in particular LUAD, more particularly stage III or IV NSCLC or LUAD.

Accordingly, the method provided herein comprises the steps of:

(i) assessing the expression level in a biological sample, preferably a tumour sample, obtained previously from a patient, of at least one miRNA selected from the group consisting of miR-24-3p, miR-23a-3p and miR-27a-3p; optionally, (ii) comparing the expression level of said miRNA with a reference level; and optionally (iv) concluding that the patient has an increased likelihood of resistance to platinum-based chemotherapy if the expression level measured in the patient is equal to or higher than said reference level.

In particular embodiments, the method additionally comprises (v) treating the patient with platinum-based chemotherapy if the expression level measured in the sample from the patient is lower than said reference level.

In particular embodiments, the method additionally comprises (v′) treating the patient with a non-platinum-based treatment if the expression level measured in the sample from the patient is equal to or higher than said reference level.

In particular embodiments, the method additionally comprises (v″) treating the patient with an inhibitor of miR-24-3p, optionally in combination with a platinum-based chemotherapy, if the expression level measured in the sample from the patient is equal to or higher than said reference level.

Accordingly, provided herein are platinum-based chemotherapeutic agents for use in patients wherein the level of expression of at least one miRNA from the miR-24-2 cluster was assessed, in particular according to any of the methods disclosed herein, and in particular wherein the expression level of at least one miRNA and/or the combined expression level of the miRNAs of interest, was found to be equal to or higher than a reference value, said value being in particular determined as provided herein.

Also provided herein is an inhibitor of miR-24-3p for use in patients wherein the level of expression of at least one miRNA from the miR-24-2 cluster was assessed, in particular according to any of the methods disclosed herein, and in particular wherein the expression level of at least one miRNA and/or the combined expression level of the miRNAs of interest, was found to be equal to or higher than a reference value, said value being in particular determined as provided herein. Such inhibitors are provided in particular for use in a treatment in combination with platinum-based chemotherapeutic agents.

A therapeutic agent is said herein to be used in combination with another therapeutic agent whenever both agents are administered to the same patient, for the treatment of the same disease (or of diseases originating from the same cause). In particular, such a use in combination comprises uses wherein the effect of both agents is meant to occur simultaneously and/or wherein the effect of one agent makes possible, facilitates and/or increases the therapeutic effect of the other agent. In particular, agents listed on the same prescription for the treatment of the same disease in a patient are considered to be used in combination. Similarly, agents the administration of which is recommended within the same treatment protocol for the treatment of a disease, in particular in treatment guidelines established by regulating agencies or medical societies, are considered to be used in combination when administered to a patient.

### Platinum-Based Chemotherapeutic Agents for Use in Treating Cancer

Provided herein is a platinum-based chemotherapeutic agent for use in a patient suffering from cancer, wherein the expression level of miR-24-3p and/or of a miRNA of the miR-24-2 cluster in a sample from said patient was assessed according to any of the methods disclosed herein and in particular wherein said level was determined to be lower than a reference value.

Methods, products, and uses of said products provided herein are intended to use for patients suffering from cancer, or patients suspected of suffering from cancer and/or patients with genetic predisposition, or living or having lived in environmental conditions favouring the development of cancer.

In some cases, the methods, products and uses thereof are part of the diagnostics process confirming that the patient does indeed suffer from cancer, in particular non-small cell lung cancer, and more particularly lung adenocarcinoma. In some embodiments, the methods, products and uses are intended to select a treatment for patients diagnosed with cancer. In some embodiments, the methods, products and uses are intended to determine, or participate in the determination, of the likelihood of response to platinum-based chemotherapy and/or of the prognosis of the patient.

Accordingly, provided herein are methods of treatment comprising the steps of assessing the expression level of a miRNA from the miR-24-2 cluster according to any of the methods disclosed herein, in particular wherein said level is determined to be lower than a reference value, concluding that the patient is likely to respond to a platinum-based chemotherapy and treating said patient with a platinum-based chemotherapeutic agent. Also provided herein are the use of a platinum-based chemotherapeutic agent for the manufacture of a medicament to treat a patient wherein the expression level of one or more miRNAs from the miR-24-2 cluster was assessed according to any of the methods disclosed herein, in particular wherein said level was determined to be lower than a reference value.

In certain embodiments, the cancer is non-small cell lung cancer (NSCLC). In certain embodiments, the cancer is lung adenocarcinoma (LUAD). In certain embodiments, the cancer is a stage III or IV NSCLC. A platinum-based chemotherapy is a treatment used for treating cancer comprising the administration of DNA intercalating and/or cross-linking and/or DNA damage-inducing chemical agents comprising at least one platinum atom. More particularly, platinum-based chemotherapeutic agents are coordination complexes of platinum, and comprise in particular cisplatin, oxaliplatin, carboplatin, nedaplatin and lipoplatin.

### MiR-24-3p Inhibitors and Compositions Comprising Such Inhibitors, in Particular for Use in the Treatment of Cancer

The overexpression of miR-24-3p has been shown by the inventors to be directly involved in the resistance to platinum-based chemotherapeutic agents. Accordingly, the inventors provide herein methods of improving response to such chemotherapeutic agents comprising inhibiting the overexpression or the effect of the overexpression of miR-24-3p.

Accordingly, provided herein is an inhibitor of miR-24-3p for use in the treatment of patients, in particular human patients with cancer, more particularly patients having an NSCLC, in particular a LUAD and more particularly stage III or IV NSCLC and/or LUAD. In particular embodiments, such an inhibitor is provided for use in improving the response to platinum-based chemotherapy in such patients.

“Improving the response to platinum-based chemotherapy” is used herein to mean obtaining an increased therapeutic effect of a said chemotherapy. The skilled person is aware of the expected therapeutic effects of such chemotherapies and of methods to monitor such effects. An improvement in such effect may be the actual observation of an effect, where none was observed prior to said improvement or where none is expected to occur without said improvement. An improvement may also consist in an increase in the magnitude of the observed effect (relative to the expected effect or to the effect observed previously).

Preferably, the inhibitor is used in the treatment of such patients in combination with a platinum-based chemotherapeutic agent. Accordingly, provided herein is a composition or a kit of compounds comprising an inhibitor of miR-24-3p and a platinum-based chemotherapy agent. In particular embodiments, such a composition or kit of compounds (hereafter simply referred to as a combination of compounds) is provided for use in the treatment of patients as disclosed herein, in particular of human patients with stage III or IV NSCLC and/or LUAD.

In certain embodiments, the combination of compounds is suitable for simultaneous administration of the miR-24-3p inhibitor and of the chemotherapeutic agent. In certain embodiments, the kit of compounds is suitable for separate administration of the miRNA-24-3p inhibitor and of the chemotherapeutic agent, in particular for administration separated in time.

Also provided herein are methods of treatment comprising the administration of a miR-24-3p inhibitor, in patients such as disclosed herein, in particular in human patients with stage III or IV NSCLC and/or LUAD. Preferably, such methods additionally comprise the administration of a platinum-based chemotherapeutic agent. In particular embodiments, the administration of the miR-24-3p inhibitor and of the chemotherapeutic agents is simultaneous. In particular embodiments, the administration of the inhibitor and the chemotherapeutic agent is separated in time.

The administration of the agents is considered herein to be simultaneous in time in particular when the two agents are administered within the same day, more particularly within the same hour. Simultaneous administration means in particular administration simultaneous in time. In particular embodiments, the administration is simultaneous in the sense that a single administration is performed, e.g. by injecting a solution comprising both agents and/or by administering orally both agents together. The administration of two agents is said to be separate when the two agents are not administered together, e.g. are administered by different routes, or in two separate injections. The administration is said to be separated in time in particular when the agents are administered at least an hour apart, and more particularly on two different days.

The methods and uses disclosed herein comprising the administration of a miR-24-3p inhibitor and a platinum-based chemotherapeutic agent may be combined with the methods disclosed comprising the assessment of the expression level of a miRNA from the miR-24-2 cluster. In particular embodiments thereof, the patient in the method of treatment comprising the administration of such inhibitor and/or the patient in whom the inhibitor is used, has been the subject of any of the testing methods disclosed herein, and in particular the expression level of a miRNA from the miR-24-2 cluster in a biological sample of said patient has been determined to be equal to or higher than a reference value.

The miR-24-3p inhibitor disclosed herein is intended to inhibit miR-24-3p activity, i.e. lower the activation level of miR-24-3p by lowering the amount of miR-24-3p present in cells and/or interfering with the activity of miR-24-3p in the cells. In particular in cells overexpressing the miR-24-3p miRNA, the inhibition of activity, in particular of the regulatory effect of miR-24-3p on genes under control of said miRNA, allows restoring the sensitivity of cells to platinum-based chemotherapeutic agents.

The agent for inhibiting the activity of miR-24-3p can be, for example, a small molecule, nucleic acid, nucleic acid analogue, peptide, protein, antibody, or variants and fragments thereof. In some embodiments, the nucleic acid agent can be DNA, RNA, nucleic acid analogue, peptide nucleic acid (PNA), pseudo-complementary PNA (pcPNA), locked nucleic acid (LNA) or analogue thereof. In some embodiments, the nucleic acid agent can be a small inhibitory RNA (RNAi), siRNA, microRNA, shRNA, miRNA, pri-miRNA and analogues and homologues and variants thereof effective in gene silencing.

In some embodiments, the agent is an oligonucleotide which comprises a nucleotide sequence which is substantially complementary to at least part of a miR-24-3p nucleotide sequence. For example, the oligonucleotide can comprise a nucleotide sequence which is substantially complementary to at least part of a pre-miR-24 nucleotide sequence, for example, miR-24-1 (accession MI0000080) and/or miR-24-2 stem-loop sequence (accession MI0000081). In some embodiments, the oligonucleotide can comprise a nucleotide sequence which is substantially complementary to at least part of the mature miR-24-3p sequence: UGGCUCAGUUCAGCAGGAACAG (SEQ ID No:1), or which is fully complementary to said sequence.

In some embodiments, the agent is an oligonucleotide which comprises a nucleotide sequence which is substantially complementary to the seed sequence of miR-24-3p. In some embodiments, the oligonucleotide comprises a nucleotide sequence which is substantially complementary to GGCUCA for miR-24-3p, see SEQ ID No 2.

In some embodiments, the oligonucleotide comprises nucleic acid modification.

Exemplary nucleic acid modifications include, but are not limited to, nucleobase modifications, sugar modifications, inter-sugar linkage modifications, backbone modifications, and any combinations thereof. Nucleic acid modifications are described below in more detail.

In some embodiments, the agent is an antagomir, fully 2′-O-methoxyethyl (2′-MOE), 2′-F/MOE mixmer, fully LNA, LNA/DNA mixmer, a tiny LNA or a combination thereof. In some embodiments, the agent is a Tiny LNA oligonucleotide. As used herein, the term “tiny LNA” refers to a short, e.g., 7, 8, 9, 10, 11 or 12-mer oligonucleotide that is comprised entirely of locked nucleic acid monomers. Tiny LNAs have been demonstrated to be effective to inhibit miRNA mediated gene suppression in vivo and are described in Obad et al., (Nature Genetics, 2010, 43(4): 371-380, content of which is incorporated herein by reference. In some embodiments, the agent is a shortmer. As used herein, the term “shortmer” refers to a short, e.g., 7, 8, 9, 10, 11 or 12-mer oligonucleotide that is comprised entirely of 2′-MOE modified nucleic acid monomers.

In some embodiments, the oligonucleotide agent can be encoded by an expression vector.

### BRIEF DESCRIPTION OF DRAWINGS

FIG. 1. High Throughput screen for miRNAs whose increased expression induces cisplatin resistance in A549 cells.

A549 cells were transfected with the pre-miR library for two days and the transfected cells were then exposed to cisplatin at 60 μM for three days. Cell viability was measured in single well using Cell Titer Glow assay.

(A): Plot of normalized viability versus −log₁₀ FDR-adjusted P-value for all miRNAs overexpressed in A549 cells. Vertical dashed lines denote the selected normalized viability cutoff, while the Horizontal line denotes the 0.01 selected FDR-adjusted P-value cutoff. Each dot represents a miRNA. Dots surrounded in the upper right quadrant represent miRNAs associated with cisplatin resistance at the chosen cutoffs. The standardized residual viability of the miR-24-3p miRNA was estimated at 4.9512.

(B) Venn diagram showing the overlap between miRNAs identified in the gain-of-function screen and those expressed in A549 cells. Expression was determined by small RNA Seq analysis on SOLiD 5500 WFTechnology). The threshold of expression was a minimum of 100 reads/millions of total mature miRNAs reads in at least one experimental condition.

FIG. 2. Effects of miR-24-3p overexpression in A549 cells on either cisplatin- or vinorelbin-induced cell death.

A549 cells were transfected with pre-miR-24-3p for 2 days and the transfected cells were then exposed to either cisplatin or vinorelbin.

(A): Annexin V/PI staining of A549 cells overexpressing miR-24-3p and exposed to 60 μM cisplatin for 24 h.

(B): Vinorelbin dose response analysis of A549 cells overexpressing miR-24-3p. An ATP-based assay was used to assess cell viability.

FIG. 3. Strategy to identify miR-24-3p targets involved in cisplatine resistance.

FIG. 4. BIM and PUMA are direct targets of miR-24-3p: Co-transfection of pre-miR-24-3p or pre-miR-Neg and either human BIM 3′UTR- or PUMA 3′UTR-derived psiCHECK-2 constructs in HEK293 cells.

(A): Sequences cloned in psiCHECK-2 in the XhoI and NotI restrictions sites

(B): Results of luciferase assays in HEK293 cells transfected with the indicated constructions and pre-miR-24-3p or control miRNA.

Cells were harvested two days after transfection and luciferase activity were analysed. All renilla luciferase activities were normalized with firefly luciferase activity. **p<0.01.

FIG. 5. DNA damage response induced by cisplatin of A549 cells overexpressing miR-24-3p.

A549 cells overexpressing miR-24-3p were exposed to cisplatin for 7 h and nuclear foci of (i) phosphorylated histone H2A.X at SER139 (y-HAX) and (ii) phosphorylated ATM at Ser1981 (p-ATM) were assessed by confocal microscopy.

(A): Representative immunofluorescence microphotographs of y-HAX,

(B): Representative immunofluorescence microphotographs of p-ATM.

(C): percentage of cells (n=3) exposed to cisplatin and containing more than five nuclear foci of y-HAX and phosphor-ATM, respectively after miR-24-3p overexpression.

FIG. 6. Western Blot showing the effects of miR-24-3p overexpression in A549 cells on (A) BIM and PUMA, (B) PNPO an PDXK, (C) the caspase 3 dependent apoptotic response induced by cisplatin.

FIG. 7. Effect of (A) BIM, (B) PUMA, (C) PDXK and (D) PNPO silencing on caspase 3 dependent apoptotic response induced by cisplatin.

FIG. 8. Effect of miR-24-3p inhibition on (A) BIM and PUMA, (B) PNPO an PDXK, (C) the caspase 3 dependent apoptotic response induced by cisplatin.

FIG. 9. Effect of TP53 mutational status on cisplatin resistance induced by miR-24-3p.

(A) Kill curve of H1299 cells (with a homozygous partial deletion of the TP53 gene) overexpressing miR-24-3p or a miRNA control and exposed to increasing dose of cisplatin.

(B) Validation of the effects of cisplatin in two LUAD cell lines, H1299 (with a homozygous partial deletion of the TP53 gene) and H1975 (homozygous for the c.818G<A mutation of the TP53 gene).

FIG. 10. In vivo experiments. miR-24-3p inhibition by LNA-modified oligonucleotides (SEQ ID Nos: 11 and 12). The results were obtained after RT-qPCR on lung total RNAs.

FIG. 11. Effects of miR-24-3p, miR-27a-3p and miR-23a-3p overexpression in A549 cells on cisplatin sensitivity.

FIG. 12. Networks of miR-24-3p, miR-27a-3p and miR-23a-3p predicted targets. Venn diagram summarizing the genes and predicted targets (in bold*) significantly down-regulated by miR-24-3p, miR-23a-3p and miR-27a-3p.

FIG. 13. Expression of miR-23a-27a-24-2 cluster and Kaplan-Meier survival curve from the TCGA data base.

Panels A, C and E: Box-plot diagram showing the expression levels of mir-24-2* (A), miR-23a (C) and miR-27a (E) measured by Small RNA-Seq in early-stages LUAD patients depending on their survival status 5 years following the diagnosis (n=293). For each plot a corresponding P value is shown. A: Alive; D: Dead.

Panels B, D, F and G: Kaplan-Meier curves for overall survival for patients with LUAD stratified by median value, according to expression of mir-24-2* (B), miR-23a (D), miR-27a (E) and the mean expression of the 3 mature miRNAs (F). The overall survival of patients with a normalized value lower than the median value is higher in all cases. For each plot the P value is indicated: (B) p-value=0.073; (D) p-value=0.0052; (F) p-value=0.02; (G) p-value=0.0365.

(*The name miR-24-2 refers to the gene, whereas the name miR-24-3p refers to the miRNA)

FIG. 14. Set up of a qPCR assay for the detection of circulating miR-23a-27a-24-2 cluster in biofluids.

(A). Summary of the miRNAs analyzed in plasmas from control patients.

(B). Correlation between the normalized expression of the selected miRNAs from the same sample in 2 independent assay plates.

(C). Correlation of miR-24 normalized expression values between the same series of 14 control plasmas analyzed in 2 independent experiments.

(D). Histogram of miR-24 normalized expression values in the series of control plasmas.

(E). Box plot showing the expression of miR-21-5p, miR-23a-3p, miR-24-3p and miR-27a-3p in the plasmas from control patients.

FIG. 15. Comparison of circulating miRNA levels in plasma from healthy donors and LUAD patients. Box plot showing the normalized expression of miR-21-5p (A), miR-23a-3p (B), miR-24-3p (C) and miR-27a-3p (D) in the plasmas from healthy donors (n=14) and LUAD patients (n=20).

### EXAMPLES

### Example 1. Identification of miRNA Associated with Cisplatin Resistance of Lung Adenocarcinoma Using Functional Genetic Screening

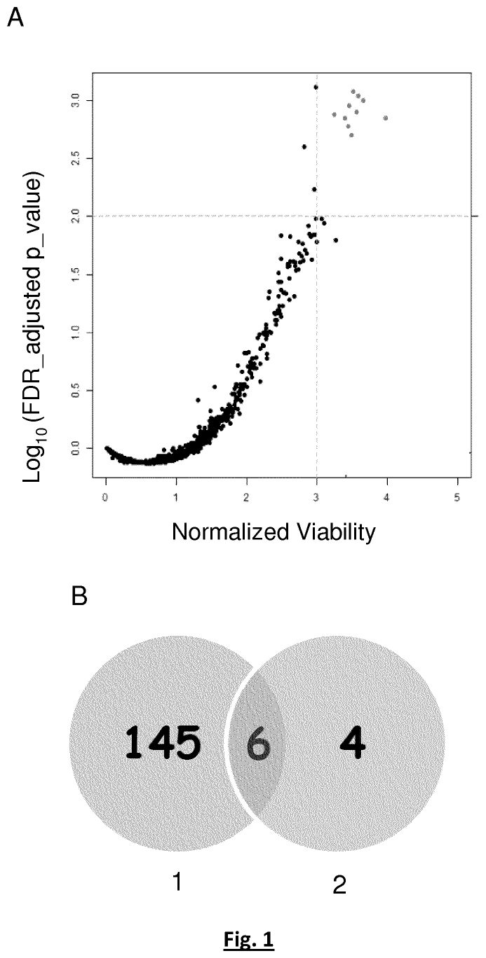

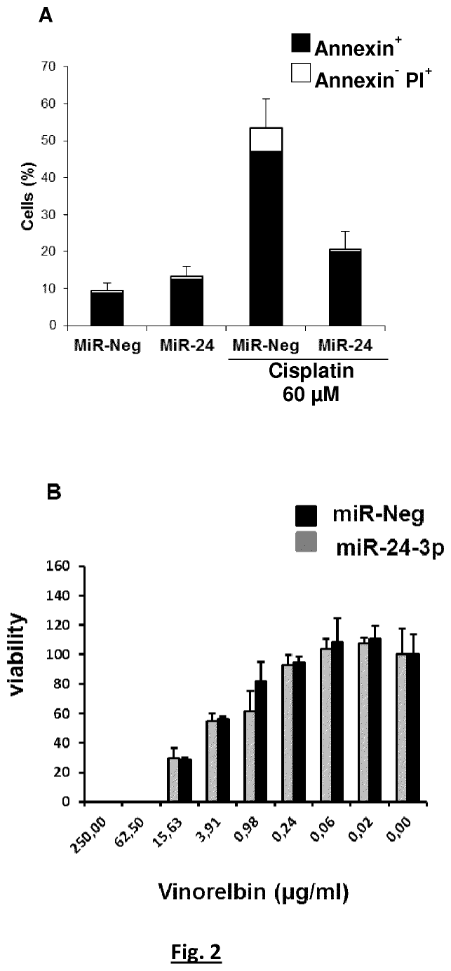

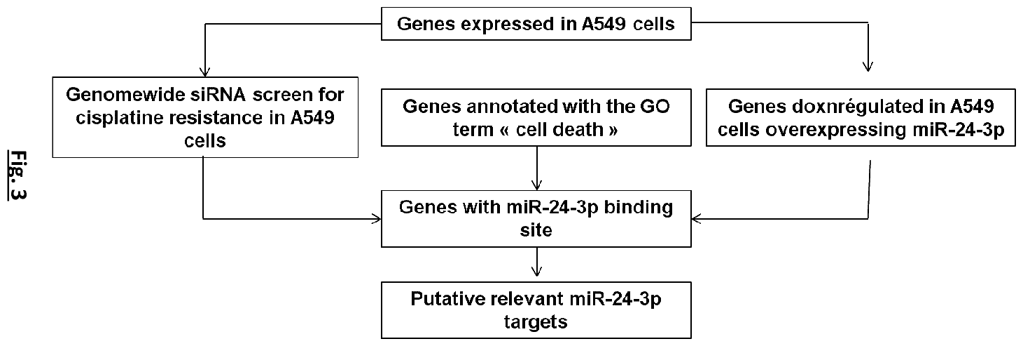

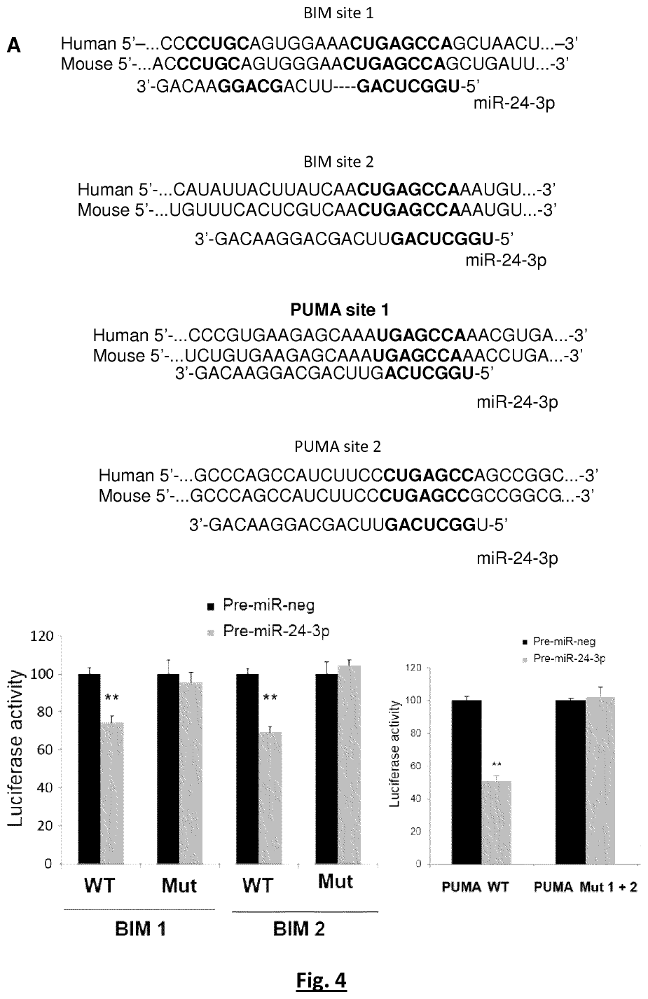

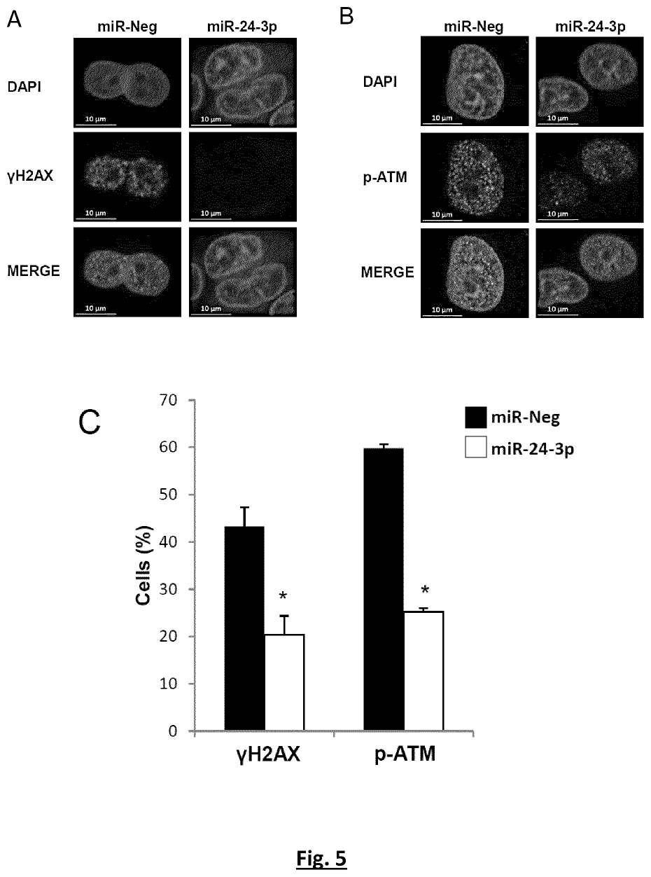

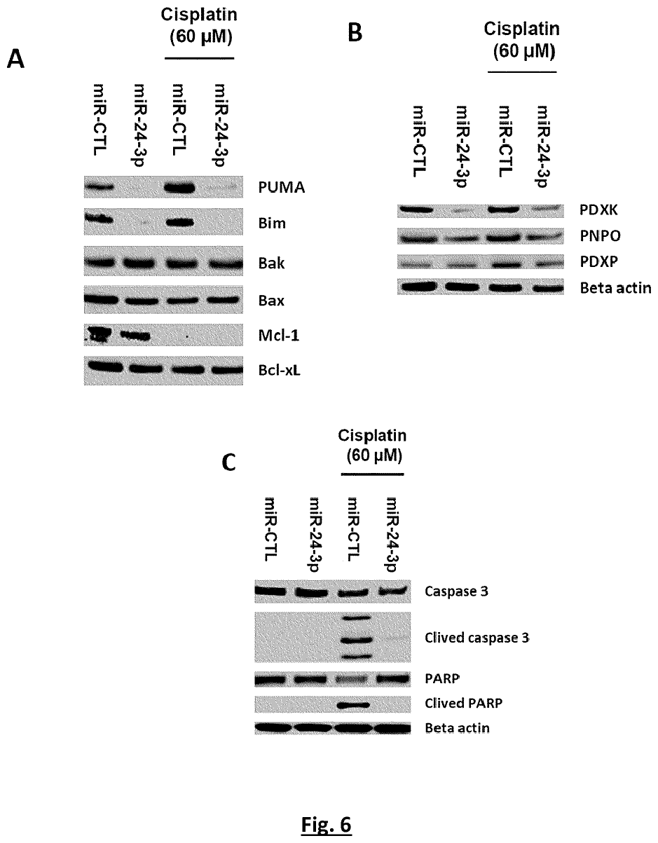

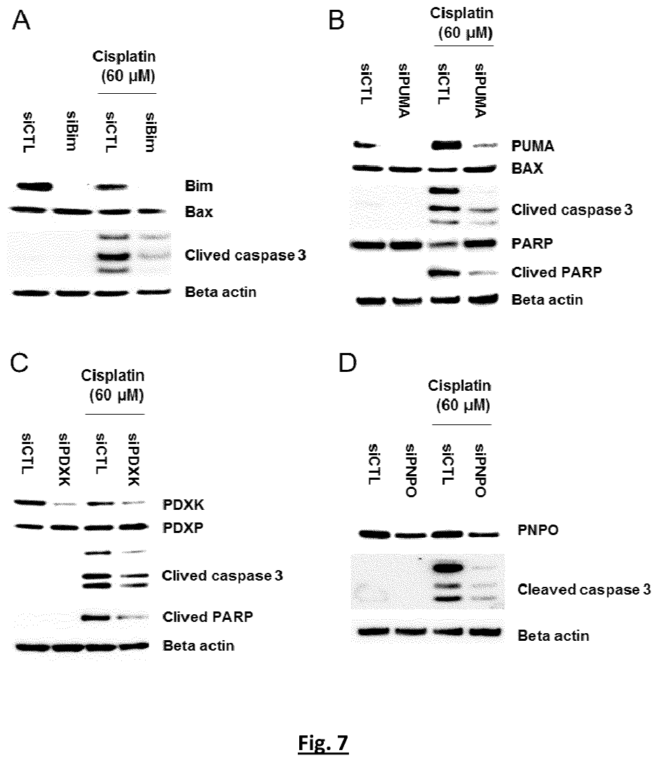

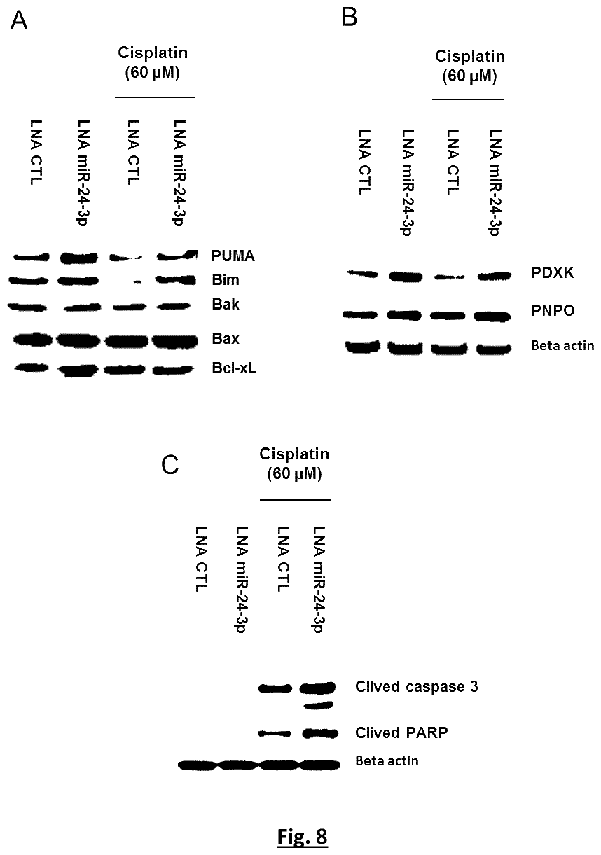

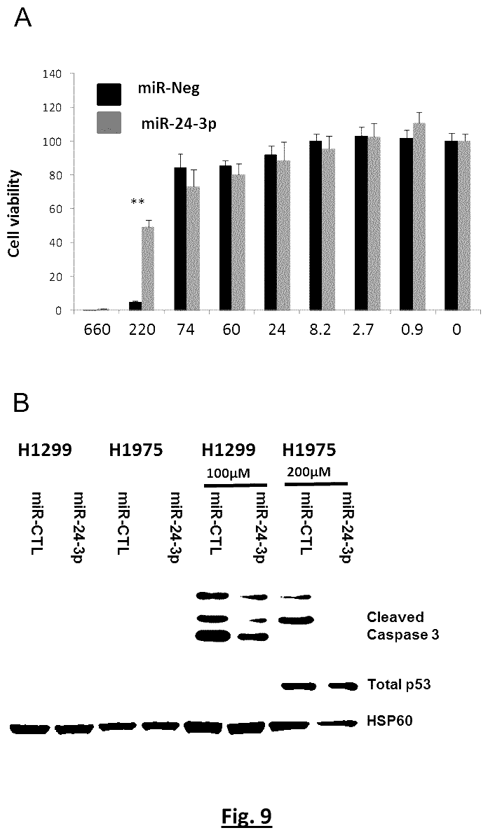

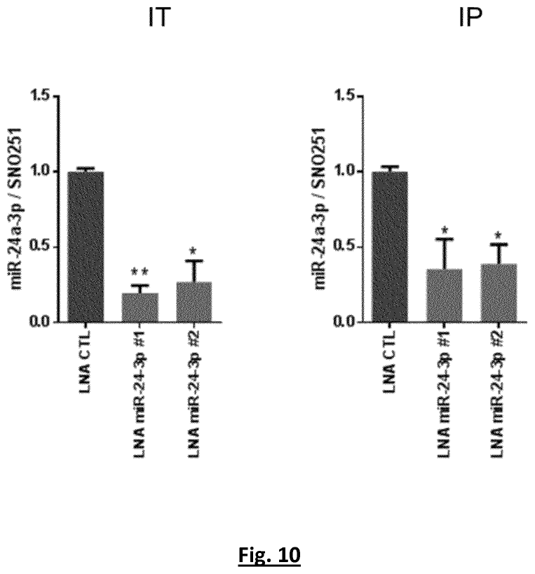

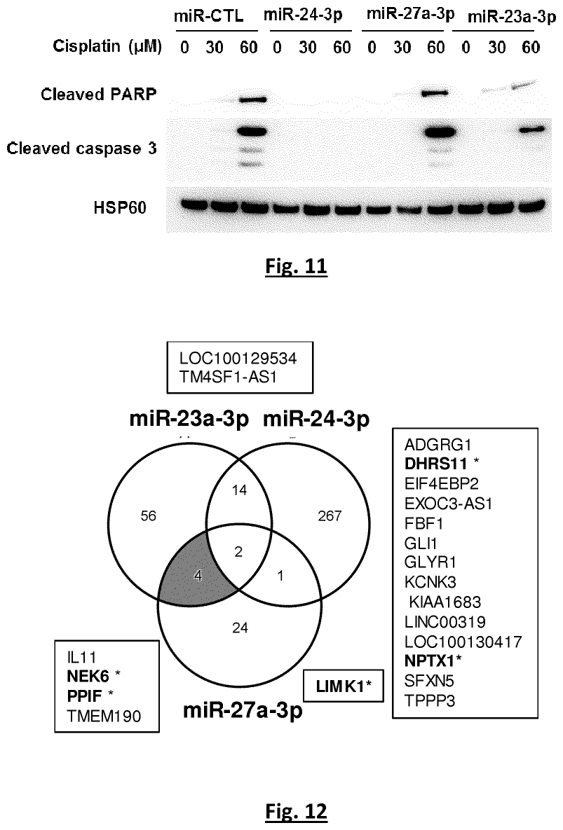

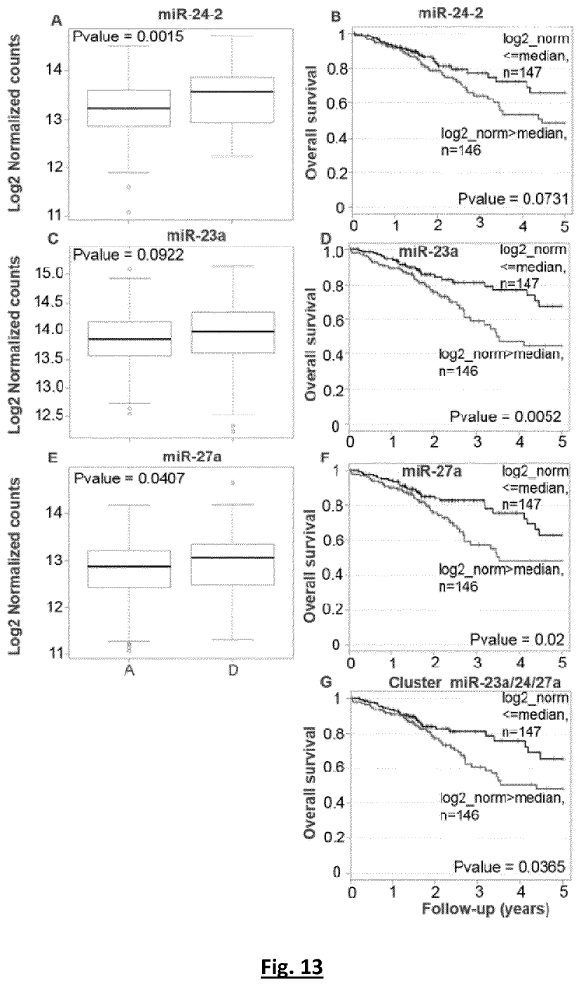

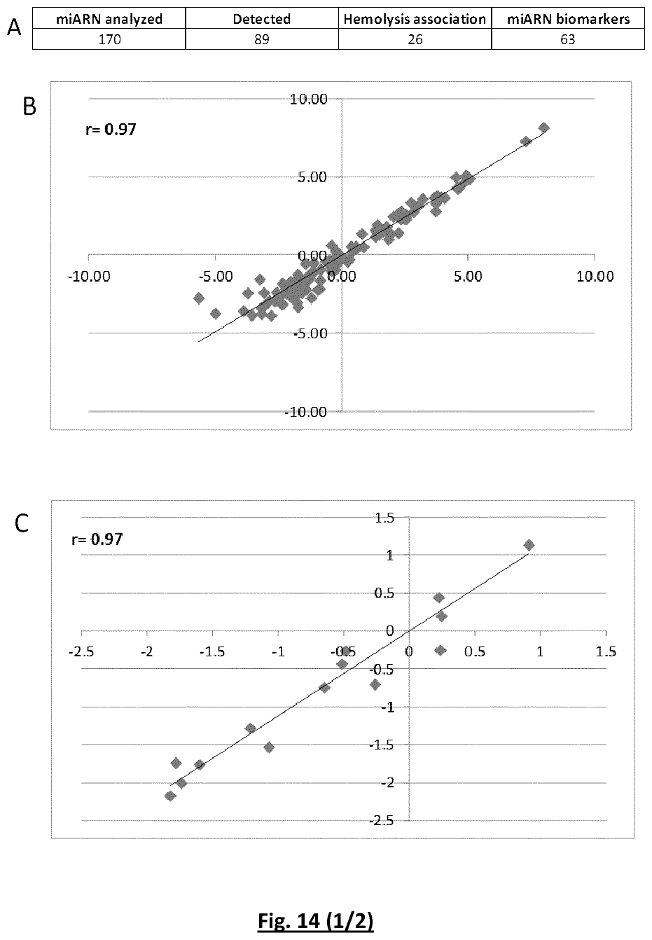

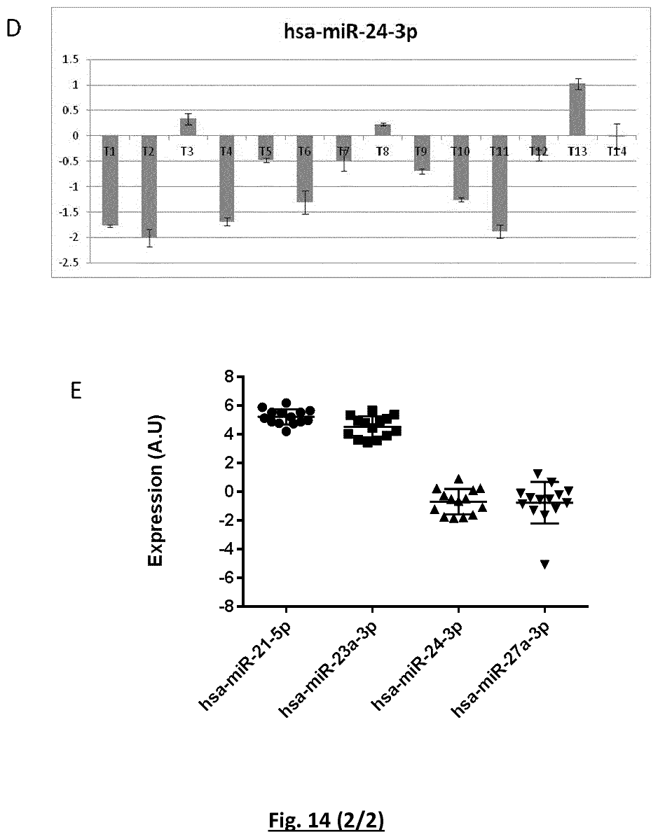

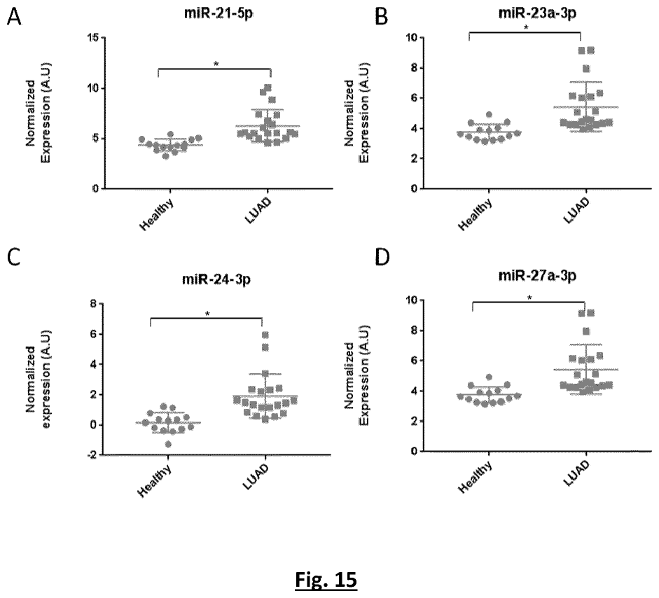

Functional genetic screen is an extremely powerful approach for cancer drug resistance gene discovery and validation (Iorns et al., 2007). Therefore the inventors used this approach to comprehensively identify miRNAs whose increased expression induce cisplatin resistance of lung adenocarcinoma cells using a library of miRNA mimics corresponding to miRbase version 16. The gain-of-function data indicates that overexpression of about 10 miRNAs reproducibly induces cisplatin resistance (2 independent screens using mimic library performed in duplicate) (FIG. 1A). Importantly, none of the miRNA identified in the screen and whose increased expression is the most associated with cisplatin resistance are currently described to affect cisplatin response of LUAD. In addition, among the 10 miRNA identified in the screen, 5 were also expressed in A549 cells (FIG. 1B). Therefore, the inventors chose to first focus on miR-24-3p, a miRNA expressed in A549 cells, and identified as the best miRNA candidate according to statistics and degree of resistance conferred by its overexpression. Importantly, miR-24-3p is also known to be upregulated in NSCLC (Zhao et al., 2015).

To ensure reliability of these findings, miR-24-3p effects on cisplatin sensitivity were independently validated in A549 cell lines (FIG. 2). These results showed that in A549 cells transfected with the pre-miR-24-3p (and so overexpressing miR-24-3p), the response to cisplatin was lesser than in cells which did not overexpress miR-24-3p. In addition, A549 cells overexpressing miR-24-3p are specifically (comparison with vinorelbin treatment) and strongly resistant to platinum based-compounds including the 2nd generation platinum analog: oxaliplatin (i.e., resistant to cisplatin, carboplatin and oxaliplatin; FIG. 2 and data not shown).

### Example 2. Identification of Relevant miR-24-3p Target Genes

To identify relevant miR-24-3p target genes, the inventors used two distinct complementary approaches (FIG. 3):

1. miR-24-3p target candidates were identified using a combination of bioinformatics and experimental approaches. For this, the inventors determined the whole gene expression profile of A549 cells overexpressing or not this miRNA and performed a bioinformatic search for genes (i) whose 3′UTR contain one (or more) miR-24-3p binding site(s) and (ii) whose annotation corresponds to GO terms associated with cell death, apoptosis, cell survival, cell cycle or senescence using tools developed by the inventors. This led the inventors to the identification and validation using luciferase constructs of BIM and PUMA, two pro-apoptotic BH3-only proteins, as miR-24-3p targets (FIG. 4).

2. A comparative analysis of data obtained through a genomewide siRNA screen study (Galluzzi et al., 2012) to identify genes (i) whose decreased expression induces cisplatin resistance in A549 cells and (ii) whose 3′UTR contains one (or more) miR-24-3p binding sites. Among the 32 genes revealed by the genomewide siRNA screening approach, only one contains miR-24-3p binding sites in its 3′UTR: PDXK, a kinase that phosphorylates vitamin B6, a central regulator of cisplatin responses (Galluzzi et al., 2012). Interestingly, another gene involved in vitamin B6 metabolism, PNPO, was also found to contain a potential miR-24-3p site in its 3′UTR and was also found repressed in the miR-24-3p signature profiling described above.

### Example 3. miR-24-3p is a Potent Regulator of Cisplatin-Induced Cell Death of LUAD

As cisplatin initiated cell death by inducing DNA lesions, the inventors explored whether overexpression of miR-24-3p in A549 cells exposed to cisplatin influenced the DNA damage response mediated by ATM before apoptosis occurred. The results of the inventors showed that A549 cells overexpressing miR-24-3p exhibited less DNA damage (assessed by phospho-yH2AX nuclear foci) as well as decreased ATM signaling 7 h following cisplatin exposure, without affecting cisplatin disposition in tumor cells (FIG. 5).

To gain further insights into the anti-apoptotic effects mediated by miR-24-3p, the inventors further studied the impact of miR-24-3p modulation on these 4 targets and the caspase 3 dependent apoptotic response induced by cisplatin (FIGS. 6-8). Altogether, these results suggest that miR-24-3p strongly inhibits caspase 3 dependent apoptotic response induced by cisplatin likely through targeting 4 genes: BIM, PUMA, PDXK and PNPO.

To assess the contribution of each miR-24-3p targets on the sensitivity of A549 cells to cisplatin, these cells were transfected with individual siRNA against either BIM, PUMA, PDXK, PNPO or a siRNA control. Expression of cleaved caspase 3 was used to evaluate the apoptotic response induced by cisplatin. Results shown on FIG. 7 demonstrated that siRNA-mediated depletion of each transcript decreased cisplatin-induced apoptosis, and thus, demonstrated that miR-24-3p promoted cisplatin resistance through direct targeting of BIM, PUMA, PDXK and PNPO.

A549 cells were transfected with miR-24-3p inhibitor or control for 24 hours and then exposed or not to cisplatin for 24 hours. The results showed that the inhibition of miR-24-3p increased the cisplatin induced apoptotic response in A549 cells, demonstrated by an increase expression of both cleaved caspase 3 and cleaved PARP, two characteristic markers of the apoptotic process (FIG. 8). These results demonstrated the possibility of using inhibitors of miR-24-3p to increase the sensitivity of resistant cells to cisplatin.

Interestingly, this data was independently validated in two other LUAD cell lines, H1299 (with a homozygous partial deletion of the TP53 gene), and H1975 (homozygous for the c.818G>A mutation of the TP53 gene) suggesting that miR-24-3p contributed to cisplatin resistance independently of p53 (FIG. 9).

### Example 4. In Vivo EXPERIMENTS

In vivo experiments were performed using 9-12 weeks old male C57BL/6 mice and LNA-modified oligonucleotides designed against miR-24-3p. These inhibitors of miR-24-3p were purchased from Exiqon: LNA miR-24-3p inhibitor n° 1 whose sequence is TGCTGAACTGAGCC (SEQ ID NO: 11) and LNA miR-24-3p inhibitor n° 2 whose sequence is GCTGAACTGAGCC (SEQ ID NO: 12).

As shown in FIG. 10, both LNA anti-miR-24-3p oligonucleotides efficiency inhibited miR-24-3p when they were administrated on the local route (intratracheal administration) but also when they were used in a systemic route of administration (intraperitoneal administration). These results demonstrated the feasibility of the in vivo miR-24-3p inhibition and suggested that these inhibitors could be used to improve the cisplatin activity in vivo.

### Example 5. Expression Levels of miRNAs of the miR-24-2 Cluster and Prognosis in LUAD Cancer Patients

The inventors collected 293 cases of LUAD patients with tumor small RNA-seq data from the TCGA database. The inventors analysed the expression of miR-24-3p in 2 subgroups of patients according to their survival status 5 years after the diagnosis and found that the overall expression differed significantly between alive and dead patients (FIG. 13, panel A). Interestingly, the inventors found similar data with the 2 other mature miRNAs expressed from the same cluster (FIG. 13, panels B and C). Kaplan-Meier survival analysis revealed that the group of patients with high expression of each of these 3 miRNAs had shorter survival compared to the low expression group (FIG. 13, panels D, E and F). These data were also confirmed using the mean expression value of the 3 miRNAs of the cluster (FIG. 13, panel G).

These data showed that a high expression level of miR-24-3p was associated with a poor prognosis in LUAD cancer patients. It might be thus clinically relevant to measure the expression levels of miR-24-3p (and of the two other members of the cluster) to stratify patients and to select patients at risk for a poor therapeutic response to cisplatin treatment.

Such an assay would comprise contacting a biological sample obtained from the subject to detect the levels of at least one (e.g., one, two, three) of miR-23a-3p, miR-24-3p and miR-27a-3p (or the mean level of these 3 miRNAs), wherein the level of expression of these miRNAs above a predefined reference level identified the subject predicted to be at risk of a poor therapeutic response.

The level of at least one miR gene product could be measured in cells of a biological sample obtained from the subject. For example, a tissue sample could be removed from a subject after lung surgery or by conventional biopsy techniques. In another example, a blood sample could be removed from the subject, and circulating RNA could be isolated by standard techniques. The blood or tissue sample could be obtained from the subject prior to initiation of any therapeutic treatment (radiotherapy or chemotherapy), preferably at time of diagnosis. A corresponding control lung tissue sample could be obtained from unaffected tissues of the subject (matched normal adjacent tissue in case of lung surgery). The control tissue or blood sample was then processed along with the sample from the subject, so that the levels of mature miRNA from the subject's sample could be compared to the corresponding levels from the control sample. An increase in the level of expression of miR-23a-3p/miR-24-3p/miR-27a-3p in the sample obtained from the subject, relative to the level of the corresponding miRNA gene product in a control sample, could be indicative of tumor aggressiveness and cisplatin resistance. The threshold used to identify LUAD patients at risk from the other could be obtained by performing expression analysis on a prospective cohort of LUAD patients with known initial response to chemotherapy (including platinum-based chemotherapeutic reagent) following the RECIST criteria. The selection of 100 patients samples (tumor tissue and matched normal adjacent tissue and plasma) is currently ongoing with the support of the Nice Hospital Tumor Biobank (Pr Paul Hofman) and expression levels of miR-23a-3p/miR-24-3p/miR-27a-3p is performed using methods described below in order to consolidate the data obtained on the TCGA cohort.

The level of a miRNA gene product in a sample could be measured using any technique that was suitable for detecting RNA expression levels in a biological sample. Suitable techniques for determining RNA expression levels in cells from a biological sample (e.g., qRT-PCR, in situ hybridization, small RNA-Seq, microarrays) are well known to those skilled in the art.

### Example 6. The miR-23a-27a-24-2 Cluster Promotes Cisplatin Resistance of LUAD

As miR-24-3p was part of a miRNA cluster transcribed as a single primary miRNA and subsequently processed into three mature miRNAs: miR-23a-3p, miR-27a-3p and miR-24-3p, the inventors evaluated whether miR-23a-3p and/or miR-27a-3p also contributed to cisplatin resistance. The results of the inventors, performed in A549 LUAD cell line, showed that, albeit to a lesser extent than miR-24-3p, overexpression of miR-23a-3p was able to induce cisplatin resistance of A549 cells (FIG. 11). Interestingly the inventors found several common predicted targets between these miRNAs, several of them having been associated with apoptotic/necrotic cell death and DNA damage such as PPIF and NEK6 (FIG. 12).

### Example 7. miR-24-3p as a Biomarker for the Prediction of Cisplatin Resistance

Analysis of data from TCGA indicated that high expression level of miR-24-3p and of the other members of the cluster was associated with a poor prognosis in LUAD cancer patients (FIG. 13). The inventors built a local prospective cohort (P. Hofman and C-H Marquette, CHU Nice) to specifically assess the predictive value of miR-24-3p expression in lung tissue or plasma from LUAD patients for the prediction of cisplatin resistance. For this goal, a multiplex qPCR protocol was set up for the detection of circulating miR-23a-27a-24-2 cluster in biofluids (FIG. 14). Data indicated that the inventors could reproducibly measure the levels of more than 60 miRNA candidates including the miR-23a-27a-24-2 cluster in plasmas from a cohort of 14 healthy donors. Preliminary data obtained on a first set of 20 serums from LUAD patients (at different stages of diagnosis) indicated that circulating levels of the miR-23a-27a-24-2 cluster were upregulated in LUAD compared to healthy donors (FIG. 15).

### Methods

RNA isolation. Total RNAs were extracted from lung tissue and cell samples with TRIzol solution (Invitrogen). Integrity of RNA was assessed by using an Agilent BioAnalyser 2100 (Agilent Technologies) (RIN above 7).

Mature miRNA expression. MiR-24-3p expression was evaluated using TaqMan MicroRNA Assay (Applied Biosystems) as specified in their protocol. Real-time PCR was performed using Universal Master Mix (Applied Biosystems) and ABI 7900HT real-time PCR machine. Expression levels of mature microRNAs were evaluated using comparative CT method (2-deltaCT). A multiplex qPCR assay on miRNA-purified plasmas was performed on a Biomark machin (Fluidigm) using the miScript Microfluidics PCR Kit (Qiagen). Normalization was performed using an external spike-in control added before RNA extraction from 200 μl of plasma.

Expression microarrays. For gene expression arrays RNA samples were labeled with Cy3 dye using the low RNA input QuickAmp kit (Agilent) as recommended by the supplier. 825 ng of labeled cRNA probe were hybridized on 8×60K high density human Agilent microarrays. Two (biological replicates were performed for each comparison. Data was log 2 transformed and normalized using a cyclic loess algorithm in the R programming environment.

Transfection. Pre-miR-24-3p, and control miRNA (miR-Neg #1) were purchased from Life technologies. For miR-24-3p knockdown experiments, LNA anti-miR-24-3p and LNA negative control were ordered from Exiqon. siRNA directed against BIM, PUMA, PNPO and PDXK were purchased from Life technologies. A549 and H1299 cells were grown in 10% FCS in DMEM and transfected at 30 to 40% confluency in 6-12- or 96 well plates using Lipofectamin RNAi MAX™ (Life technologies) with pre-miRNA, siRNAs LNA inhibitors at a final concentration of 5 nM unless indicated.

Luciferase assay. Molecular constructs were made in psiCHECK-2 (Promega) by cloning behind the Renilla luciferase in the XhoI and NotI restrictions sites, annealed oligonucleotides derived from BIM, PUMA 3′ UTR. HEK293 cells were plated into 96-well and cotransfected using lipofectamin 2000 (Invitrogen) with 0.2 μg of psiCHECK-2 plasmid construct and pre-miR-24-3p or control miRNA at different concentrations. 48 hours after transfection, Firefly and Renilla Luciferase activities were measured using the Dual-Glo Luciferase assay (Promega).

Annexin V assay. Annexin-V-FITC apoptosis detection kit (Life technologies) was used to detect apoptotic activity. Cells were collected and resuspended in binding buffer, and incubated with annexin-V-FITC and propidium iodide in the dark for 15 minutes. Annexin-V-FITC binding was determined by flow cytometry (excitation wavelength of 488 nm; emission wavelength of 530 nm) using the FITC signal detector (FL1), and propidium iodide staining was detected by the phycoerythrin emission signal detector (FL2).

Functional genetic screen. Mimics were transfected in authenticated A549 lung adenocarcinoma cell line in 96 well plate format. 48 h following transfection, cells were exposed to cisplatin at 60 μM (2 times DL50; DL50 corresponding to the drug concentration lethal to 50% of the tumor cells) for three days. Viability was assessed using the cell titer glo assay (Promega). Normalization was carried out by dividing each sample value by the median of all samples on the plate (the majority of sample wells will thus serve as a reference). Hits were identified using the rank-product method. Normalization and statistics will be performed using the R software environment.

miRNA candidate selection. miRNA candidates were selected for further analysis based on statistical significance (p<0.01) and degree of resistance induced (assessed by viability after drug exposure, normalized viability above 3).

Functional genetic screen validation for miR-24-3p. Data were confirmed independently on a distinct lung adenocarcinoma cell line (H1299). Specificity to cisplatin resistance was assessed using docetaxel and vinorelbine.

MiRNA targets analysis. MiRonTop is an online java web tool (available athttp://www.microarray.fr:8080/miRonTop/index) that integrates DNA microarrays data to identify the potential implication of miRNAs on a specific biological system (Lebrigand et al., 2010). Briefly, MiRonTop ranks the transcripts into 2 categories (‘Upregulated’ and ‘Downregulated’), according to thresholds for expression level and for differential expression. It then calculates the number of predicted targets for each miRNA, according to the prediction software selected (Targetscan, MiRBase, PicTar, exact seed search: 2-7 or 1-8 first nucleotides of the miRNA, TarBase v1), in each set of genes. Enrichment in miRNA targets in each category is then tested using the hypergeometric function.

Protein extraction and immunoblotting. Cells were lysed in lysis buffer (M-PER protein extraction reagent) and protease inhibitors cocktail (Pierce). The lysates were quantified for protein concentrations using the Bradford assay (Biorad). Proteins (10 μg per sample) were separated by SDS-polyacrylamide gel and transferred onto nitrocellulose membranes (GE Healthcare). The membranes were blocked with 5% fat free milk in Tris-buffered saline (TBS) containing 0.1% Tween-20 (TBS-T) and subsequently incubated with primary antibodies overnight at 4° C. After washing with TBS-T for 30 minutes at room temperature, the membrane was further incubated with horseradish peroxidase-conjugated secondary antibodies for 1.5 hours, followed by 30 minutes of washing with TBS-T. Protein bands were visualized with Amersham ECL substrates (GE Healthcare).

Immunofluorescence analysis. A549 cells were grown on a Round Glass Coverslips Ø16 mm (thermo scientific) placed inside a 12 Multiwell Plate. Coverslips slides were washed in phosphate-buffered saline and fixed in 4% paraformaldehyde for 15 min, cells were then permeated using 0.1% Triton X-102 (Agilent Technologies) for 10 min. and blocked with PBS solution containing BSA (3%) for 30 min. Incubation with primary antibodies was performed in a blocking solution BSA (1%) at 37° C. for 1 h. After three washes with PBS, cells were incubated with secondary antibodies. Forty five min later, Coverslips slides were fixed on microscope slides using ProLong Gold Antifade Reagent with DAPI (Invitrogen). Fluorescence was viewed with an FV10i Olympus confocal scanning microscope.

Quantification of intracellular platinum. For total cell platinum accumulation experiments, cells were lyzed in RIPA lysis buffer after extensive washing with ice-cold PBS and allowed to settle in the incubator overnight. Intracellular quantification of platinum was determined by atomic absorption spectrometry (AA spectrometer). Platinum levels were normalized to protein levels.

Detection of cisplatin-GG DNA adducts. Immunofluorescence staining and measurement of specific DNA platination products was performed essentially as previously described (Liedert et al., Nucleic Acids Res, 2006, 34, e47) using a rat primary antibody that specifically recognizes CDDP-GG DNA adducts (RC-18).

In vivo experiments were performed using 9-12 weeks old male C57BL/6 mice purchased from Charles River. LNA-modified oligonucleotides designed against miR-24-3p were purchased from Exiqon: LNA miR-24-3p inhibitor no 1 whose sequence is TGCTGAACTGAGCC (SEQ ID NO: 11) and LNA miR-24-3p inhibitor no 2 whose sequence is GCTGAACTGAGCC (SEQ ID NO: 12). Said LNA miR-24-3p inhibitors were dissolved in PBS and then injected intratracheally using a MicroSprayer Aerosolizer (single dose of 5 mg/kg) or intraperitoneally using an insulin syringe (single dose of 10 mg/kg). Three days after miR-24-3p inhibitor injection, lungs were collected and total RNAs were extracted using the phenol-chloroform method.

RT qPCR: miRNA expression was assessed using TaqMan MicroRNA Reverse Transcription Kit and TaqMan MicroRNA Assays (Thermo Fisher Scientific) as specified by the manufacturer. Real-time PCR was performed using Universal Master Mix II (Thermo Fisher Scientific) and ABI 7900HT real-time PCR machine. Expression levels of mature microRNAs were evaluated using comparative CT method. For normalization, transcript levels of SNO251 (mouse samples) were used as endogenous control for miRNA real time PCR.

### REFERENCES

Chang A. Chemotherapy, chemoresistance and the changing treatment landscape for NSCLC.Lung Cancer. 2011. 71: 3-10.
Friboulet L et al. ERCC1 isoform expression and DNA repair in non-small-cell lung cancer. N Engl J Med. 2013. 368: 1101-10.
Galluzzi L et al. Prognostic impact of vitamin B6 metabolism in lung cancer. Cell Rep. 2012. 2:257-69
Goldstraw P et al. Non-small-cell lung cancer. Lancet. 2011. 378: 1727-40.
Iorns E, et al. Utilizing RNA interference to enhance cancer drug discovery. Nat Rev Drug Discov. 2007. 6: 556-68.
Jemal et al. Combinatorial microRNA target predictions. Nat Genet 2005. 37: 495-500.
Le Brigand K et al. MiRonTop: mining microRNAs targets across large scale gene expression studies. Bioinformatics. 2010. 26: 3131-2.
Zhao G, et al. Upregulation of miR-24 promotes cell proliferation by targeting NAIF1 in non-small cell lung cancer. Tumour Biol. 2015. 36:3693-701

1. An inhibitor of miR-24-3p, comprising a locked nucleic acid, for use in the treatment of a human patient with lung cancer.

2. An inhibitor of miR-24-3p for use according to claim 1, wherein said patient has non-small cell lung cancer (NSCLC), lung adenocarcinoma (LUAD) and stage III or IV NSCLC and/or LUAD.

3. An inhibitor of miR-24-3p for use according to claim 1, for use in combination with a platinum-based chemotherapeutic agent.

4. An inhibitor of miR-24-3p of claim 1, for use in improving the response to platinum-based chemotherapy in a human patient with lung cancer, a human patient with non-small cell lung cancer (NSCLC) and/or lung adenocarcinoma (LUAD) and stage III or IV NSCLC and/or LUAD.

5. A combination of compounds comprising an inhibitor of miR-24-3p of claim 1, and a platinum-based chemotherapy agent.

6. A combination of compounds according to claim 5, wherein said combination of compounds is suitable for simultaneous administration of the miR-24-3p inhibitor and of the chemotherapeutic agent.

7. A combination of compounds according to claim 5, wherein said combination of compounds is suitable for administration separated in time of the miR24-3p inhibitor and of the chemotherapeutic agent.

8. A combination of compounds according to claim 5, for use in the treatment of a human patient with lung cancer, a human patient with NSCLC and/or LUAD and stage III or IV NSCLC and/or LUAD.

9. An in vitro method of determining likelihood of response to platinum-based chemotherapy in a human patient with lung cancer, comprising the step of assessing the level of expression of at least one miRNA selected from the group consisting of miR-24-3p, miR-23a-3p and miR-27a-3p in a biological sample previously obtained from a patient.

10. A method according to claim 9, wherein the patient has non-small cell lung cancer (NSCLC), lung adenocarcinoma (LUAD).

11. A method according to claim 10, wherein the patient has stage III or IV NSCLC and/or LUAD.

12. A method according to claim 9, wherein said biological sample is a sample from a tumour of said patient or wherein said biological sample is a blood sample from said patient.

13. A method according to claim 9, wherein the expression level is assessed for one, two, or three miRNAs selected from the group consisting of miR-24-3p, miR-23a-3p and miR-27a-3p.

14. A method according to claim 9, wherein the expression level of said miRNA(s) is measured using at least one method selected from the group consisting of quantitative PCR (qPCR), real-time qPCR and expression microarrays.

15. A method according to claim 9, wherein the expression level of said miRNA(s) is compared to a preestablished reference level, said reference level being the average expression level or the median expression level measured in a group of patients having LUAD cancer, wherein the patients in said group are either unselected for response to platinum-based chemotherapy or are selected non-responder patients.

16. A method according to claim 9, wherein the expression level of said miRNA(s) in a biological sample from a tumour of said patient is compared with a reference level which is the expression level in a biological sample from non-tumour tissue of said patient.

17. A method according to claim 9, wherein the expression level of at least two miRNAs selected from the group consisting of miR-24-3p, miR-23a-3p and miR-27a-3p, and optionally wherein the average expression level of the miRNAs is used for comparisons.

18. A method according to claim 9, additionally comprising the step of concluding that the patient has an increased likelihood of resistance to platinum-based chemotherapy if the expression level measured in the biological sample of said patient is equal to or higher than said reference level.

19. (canceled)
